(19) United States
(12) Patent Application Publication
(10) Pub. No.: US 2020/0281870 A1
(43) Pub. Date: Sep. 10, 2020

(54) COMPOSITION AND USE THEREOF

(71) Applicant: Richard Baybutt, Winterville, NC (US)

(72) Inventor: Richard Baybutt, Winterville, NC (US)

(21) Appl. No.: 16/884,681

(22) Filed: May 27, 2020

Related U.S. Application Data
(62) Division of application No. 16/329,677, filed on Feb. 28, 2019.
(60) Provisional application No. 62/382,198, filed on Aug. 31, 2016.

(51) Int. Cl. A61K 31/07; A24F 47/00; A24B 15/34; A24B 15/40; A24B 15/167; A24D 3/14; A24B 15/30; A23L 33/15; A24B 15/00; A24D 3/04; A24D 1/00; A61P 9/00; A61P 11/06; A61P 35/00; A61K 9/00; A61K 31/355

(52) U.S. Cl. CPC A61K 31/07; A61K 31/355; A24B 15/34; A24B 15/403; A24B 15/167; A24D 3/14; A24B 15/30; A23L 33/15; A24B 15/00; A24D 3/048; A24D 1/002; A61P 9/00; A61P 11/06; A61P 35/00; A61K 9/0078; A24F 47/00

(57) ABSTRACT

A composition that includes vitamin A (e.g., retinol) and vitamin E (e.g., gamma-tocopherol), suitable for at least one of pulmonary administration, aerosolization, and intranasal administration. The composition can further include a pharmaceutically acceptable solid or liquid carrier. The composition can be in a unit dosage form or a metered dosage form and can be configured for pulmonary or intranasal administration. Also included are nutritional supplements, intranasal sprays, pulmonary sprays, pulmonary powders, cigarettes, and/or electronic cigarettes, each including the composition. Also included are methods of administering the composition to a human, e.g., within the oral cavity or within the respiratory system.

### CROSS-REFERENCE TO RELATED APPLICATIONS

This application is a divisional of U.S. patent application Ser. No. 16/329,677 filed on Feb. 28, 2019, which is a U.S. National Filing under 35 U.S.C. § 371 of International Patent Appl. Ser. No. PCT/US2017/049623, filed on Aug. 31, 2017, and published on Mar. 8, 2018, as WO 2018/045174 A1, which claims the benefit of U.S. Appl. Ser. No. 62/382,198, filed Aug. 31, 2016, all of which are incorporated by reference as if fully set forth herein.

### BACKGROUND

Cigarette smoking harms nearly every organ of the body, causes many diseases, and reduces the health of smokers in general.^{1,2} Cigarette smoking causes more than 480,000 deaths each year in the United States. This is nearly one in five deaths.^{1,2,3} Smoking can cause cancer almost anywhere in your body including:^{1,2} bladder, blood (acute myeloid leukemia), cervix, colon and rectum (colorectal), esophagus, kidney and ureter, larynx, liver, oropharynx (includes parts of the throat, tongue, soft palate, and the tonsils), pancreas, stomach, trachea, bronchus, and lung. Smoking also increases the risk of dying from cancer and other diseases in cancer patients and survivors.¹

In addition, smoking can cause lung disease by damaging your airways and the small air sacs (alveoli) found in your lungs.^{1,2} Lung diseases caused by smoking include COPD, which includes emphysema and chronic bronchitis.^{1,2} Cigarette smoking causes most cases of lung cancer.^{1,2} If one has asthma, tobacco smoke can trigger an attack or make an attack worse.^{1,2} Smokers are 12 to 13 times more likely to die from COPD than nonsmokers.¹ Smokers are more likely than nonsmokers to develop heart disease, stroke, and lung cancer.

Smoking is estimated to increase the risk for coronary heart disease by 2 to 4 times^{1,4}; for stroke by 2 to 4 times¹; of men developing lung cancer by 25 times¹; of women developing lung cancer by 25.7 times.¹

Smoking causes diminished overall health, increased absenteeism from work, and increased health care utilization and cost.¹

Smoking cessation (colloquially quitting smoking) is the process of discontinuing tobacco smoking. Tobacco contains nicotine, which is addictive, making the process of quitting often very prolonged and difficult. Because nicotine is addictive, quitting smoking leads to symptoms of nicotine withdrawal such as craving, anxiety and irritability, depression, and weight gain.⁵

### BRIEF DESCRIPTION OF THE FIGURES

FIG. 1 is a schematic diagram of a cigarette, according to an embodiment.

### SUMMARY

The composition described herein surprisingly has a utility for humans afflicted with, diagnosed with, or at risk of developing, a tobacco smoke pathology. For example, the composition described herein is unexpectedly believed to effectively (i) protect a human against one or more tobacco smoke pathologies, (ii) decrease in a human, the occurrence of one or more diseases caused by tobacco smoking, and/or (iii) decrease in a human, the progression of one or more diseases caused by tobacco smoking. Specifically, the human can be afflicted with, or is at risk of developing, one or more of (at least partially caused by tobacco smoking): (i) emphysema, (ii) COPD, (iii) bronchitis, (iv) pneumonitis or pulmonitis, (v) lung cancer, (vi) oral cancer, (vii) tongue cancer, and (viii) heart disease.

Embodiments are directed to a composition that includes (i) vitamin A (e.g., retinol) and esters thereof (e.g., retinol palmitate) and (ii) vitamin E (e.g., gamma-tocopherol), that is suitable for at least one of pulmonary administration, aerosolization, and intranasal administration, or any isoform of vitamin E or combination thereof. The examples described herein relate to retinol and gamma-tocopherol, but, in some embodiments include a combination of retinol and at least one of any form of vitamin E (e.g., alpha-, beta-, gamma- and delta-tocopherol) and tocotrienol (e.g., alpha-, beta-, gamma-, and deta-tocotrienol) or any combination of different forms of vitamin E and/or tocotrienol along with the retinol and/or esters thereof, including:

| Form | Structure |
|---|---|
| alpha-Tocopherol | 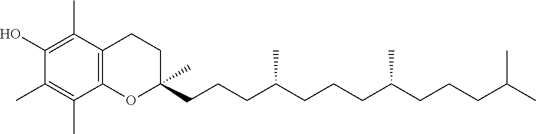 [SMILES: Cc1c(C)c2c(c(C)c1O)CC[C@@](C)(CCC[C@H](C)CCC[C@H](C)CCCC(C)C)O2] |
| beta-Tocopherol | 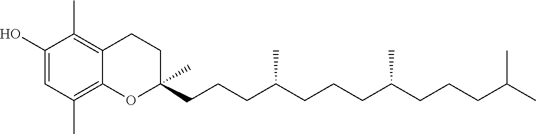 [SMILES: Cc1cc(O)c(C)c2c1O[C@](C)(CCC[C@H](C)CCC[C@H](C)CCCC(C)C)CC2] |
| gamma-Tocopherol | 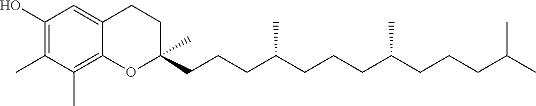 [SMILES: Cc1c(O)cc2c(c1C)O[C@](C)(CCC[C@H](C)CCC[C@H](C)CCCC(C)C)CC2] |
| delta-Tocopherol | 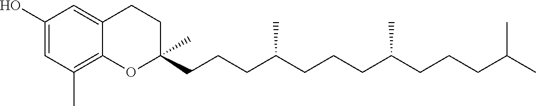 [SMILES: Cc1cc(O)cc2c1O[C@](C)(CCC[C@H](C)CCC[C@H](C)CCCC(C)C)CC2] |

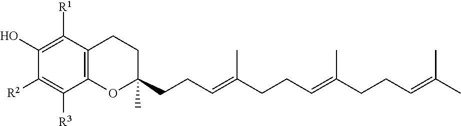

General chemical structure of tocotrienols. alpha(α)-Tocotrienol: R1 = Me, R2 = Me, R3 = Me; beta(β)-Tocotrienol: R1 = Me, R2 = H, R3 = Me; gamma(γ)-Tocotrienol: R1 = H, R2 = Me, R3 = Me; delta(δ)-Tocotrienol: R1 = H, R2 = H, R3 = Me

Embodiments are also directed to a method that includes administering to a human an effective amount of a composition of vitamin A (e.g., retinol) and vitamin E (e.g., gamma-tocopherol).

Embodiments are also directed to a nutritional supplement that includes a composition of vitamin A (e.g., retinol) and vitamin E (e.g., gamma-tocopherol), in a unit dosage form or a metered dosage form.

Embodiments are also directed to a intranasal spray that includes a composition of vitamin A (e.g., retinol) and vitamin E (e.g., gamma-tocopherol) and a pharmaceutically acceptable liquid carrier, suitable for intranasal administration.

Embodiments are also directed to a pulmonary spray that includes a composition of vitamin A (e.g., retinol) and vitamin E (e.g., gamma-tocopherol) and a pharmaceutically acceptable liquid carrier, suitable for pulmonary administration.

Embodiments are also directed to a pulmonary powder that includes a composition of vitamin A (e.g., retinol), vitamin E (e.g., gamma-tocopherol) and a pharmaceutically acceptable solid carrier, suitable for pulmonary administration.

Embodiments are also directed to a cigarette that includes a composition of vitamin A (e.g., retinol) and vitamin E (e.g., gamma-tocopherol), suitable for pulmonary administration.

Embodiments are also directed to a liquid or solid composition of vitamin A (e.g., retinol) and vitamin E (e.g., gamma-tocopherol), suitable for contacting a cigarette.

Embodiments are also directed to an electronic cigarette that includes a handheld electronic device configured to effectively vaporize a flavored liquid that is suitable for pulmonary administration, wherein the flavored liquid includes a composition of vitamin A (e.g., retinol) and vitamin E (e.g., gamma-tocopherol), and one or more of nicotine, propylene glycol, glycerin, cannabinoids, and a flavoring agent.

Embodiments are also directed to a cigarette that includes: (i) tobacco leaves dried to a water content of less than about 5 wt. %, (ii) rolling paper, (iii) optionally a cigarette filter, and (iv) a composition of vitamin A (e.g., retinol) and vitamin E (e.g., gamma-tocopherol), which is in contact with at least one of the dried tobacco leaves, the rolling paper, and the cigarette filter, wherein the rolling paper substantially forms a cylinder with the dried tobacco leaves located inside the cylinder and wherein the cigarette filter, when present, is located at one terminus of the cylinder.

Embodiments are also directed to a cigarette that includes: (i) marijuana leaves dried to a water content of less than about 5 wt. %, (ii) rolling paper, (iii) optionally a cigarette filter, and (iv) a composition of vitamin A (e.g., retinol) and vitamin E (e.g., gamma-tocopherol), which is in contact with at least one of the dried marijuana leaves, the rolling paper, and the cigarette filter, wherein the rolling paper substantially forms a cylinder with the dried marijuana leaves located inside the cylinder and wherein the cigarette filter, when present, is located at one terminus of the cylinder.

Embodiments are also directed to a method that includes: (i) igniting a terminus of the cigarette described herein, the terminus including at least one of the rolling paper and the dried tobacco, the terminus not including the cigarette filter, (ii) directing tobacco smoke from the ignited cigarette into the mouth, and (iii) releasing the tobacco smoke, wherein the presence of a composition of vitamin A (e.g., retinol) and vitamin E (e.g., gamma-tocopherol) decreases the occurrence of one or more diseases caused by tobacco smoking.

### DESCRIPTION

Reference will now be made in detail to various embodiments illustrated herein. While the embodiments will be described in conjunction with the enumerated claims, it will be understood that the embodiments disclosed herein do not limit the claims. On the contrary, the disclosed embodiments are intended to cover alternatives, modifications, and equivalents, which may be included within the scope of the claims.

References in the specification to “one embodiment”, “specific embodiments”, “an example embodiment”, etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Embodiments are directed to a composition that includes vitamin A (e.g., retinol) and vitamin E (e.g., gamma-tocopherol). The composition can further include a pharmaceutically acceptable solid or liquid carrier. The composition can be formulated, e.g., in a unit dosage form or a metered dosage form and can be configured, e.g., for pulmonary or intranasal administration. Also included are nutritional supplements, intranasal sprays, pulmonary sprays, pulmonary powders, cigarettes, electronic cigarettes, etc., each including the composition. Also included are methods of administering the composition to a human, e.g., within the oral cavity or within the respiratory system (e.g., intranasal or pulmonary).

### Composition

The compositions described herein include vitamin A (e.g., retinol) and vitamin E (e.g., gamma-tocopherol). The composition can exist in any suitable and effective formulation type, dosage form, and for any suitable route of administration. For example, the composition can exist in a solid, powder form, suitable for oral or pulmonary administration. The composition can also exist in a liquid form, suitable for aerosolization, intranasal administration, or pulmonary administration. Additionally, the composition can exist in a gaseous (or vapor) form, suitable for aerosolization, intranasal administration, or pulmonary administration.

The composition can exist as a nutritional supplement in a unit dosage form or a metered dosage form. The composition can also exist as a liquid spray, suitable for intranasal administration. The composition can also exist as a liquid spray, suitable for pulmonary administration. The composition can also exist as a gaseous (vapor) spray, suitable for pulmonary administration. The composition can also exist as a solid powder, suitable for pulmonary administration. The composition can also exist as a cigarette, suitable for pulmonary administration. Additionally, the composition can exist as an electronic cigarette, suitable for pulmonary administration.

The composition can optionally further include one or more additional substances. Such substances include, e.g., pharmaceutically acceptable carriers and/or pharmaceutically acceptable excipients. The nature and amount of such substances will depend upon, e.g., the specific formulation type, dosage form and route of administration of the composition, as well as the intended method of use. For example, when the composition is a liquid, present in an electronic cigarette to treat a human afflicted with a tobacco smoke pathology, the additional substances can include, e.g., nicotine, propylene glycol, glycerin, and a flavoring agent.

Pharmaceutically acceptable carriers and excipients suitable for the compositions described herein are well-known to those of skill in the art of pharmaceutical formulations and development, as are the general methods to manufacture the compositions described herein that include such substances. Such pharmaceutically acceptable carriers and excipients suitable for the compositions of the present, as well as the general methods to manufacture the compositions described herein that include such substances, are readily available from standard treatises, such as, e.g., Handbook of Pharmaceutical Excipients, Pharmaceutical Press, 6th ed. (2009); Remington's Pharmaceutical Sciences, Mack Publishing, 18^{th} ed. (1990); and The Theory and Practice of Industrial Pharmacy, Stripes Publ., 3^{rd} ed. (1986); which are incorporated by reference herein their entirety.

In metered dosage inhalers (MDIs), the composition will typically be stored in solution in a pressurized canister that contains a propellant, although it may also be a suspension. The MDI canister will typically be attached to a plastic, hand-operated actuator. On activation, the metered-dose inhaler releases a fixed dose of the composition in aerosol form. The correct procedure for using an MDI is to first fully exhale, place the mouth-piece of the device into the mouth, and having just started to inhale at a moderate rate, depress the canister to release the composition. The aerosolized composition is drawn into the lungs by continuing to inhale deeply before holding the breath for 10 seconds to allow the aerosol to settle onto the walls of the bronchitis and other airways of the lung. To reduce deposition in the mouth and throat, and to reduce the need for precise synchronization of the start of inhalation with actuation of the device, MDIs are sometimes used with a complementary spacer or holding chamber device.

Dry powder inhalers typically release a metered or device-measured dose of powdered composition that is inhaled through a DPI device. Nebulizers typically supply the composition as an aerosol created from an aqueous formulation.

### Methods of Use

The compositions described herein have a utility for humans afflicted with, or at risk of developing, a tobacco smoke pathology. As such, the compositions described herein can be administered to a human afflicted with, or at risk of developing, a tobacco smoke pathology. The human can be at least 18 years old, as the legal age to purchase tobacco products in the U.S. is currently 18 years. However, given that exposure to second hand smoke presents health issues, the human can be younger than 18 years old.

Given the utility of the composition for humans afflicted with, or at risk of developing, a tobacco smoke pathology, the composition can specifically be administered to a human that currently smokes tobacco. Such a human (typically referred to as a “smoker”) is considered to currently engage in the practice of taking tobacco smoke into the mouth, and then releasing it. The tobacco smoke can be inhaled, or not.

Additionally, given the utility of the composition for humans afflicted with, or at risk of developing, a tobacco smoke pathology, the composition can specifically be administered to a human that previously smoked tobacco. Such a human (typically referred to as a “smoker” or former “smoker”) is considered to have previously engaged in the practice of taking tobacco smoke into the mouth, and then releasing it. The tobacco smoke could have been inhaled, or not. The period of time for the smoking could have occurred over an extended period of time (e.g., at least about 1 year, at least about 5 years, at least about 10 years, at least about 15 years, at least about 20 years, or at least about 25 years). Additionally, the amount of the smoking could have included, on average, multiple cigarettes (e.g., at least about 5 cigarettes, at least about 10 cigarettes, or at least about 1 pack) each day.

For example, the composition described herein is believed to be able to effectively (i) protect a human against one or more tobacco smoke pathologies, (ii) decrease in a human, the occurrence of one or more diseases caused by tobacco smoking, and/or (iii) decrease in a human, the progression of one or more diseases caused by tobacco smoking. Specifically, the human can be afflicted with, diagnosed with, or is at risk of developing, one or more of (at least partially caused by tobacco smoking): (i) emphysema, (ii) COPD, (iii) bronchitis, (iv) pneumonitis or pulmonitis, (v) lung cancer, (vi) oral cancer, (vii) tongue cancer, and (viii) heart disease.

### Cigarettes and Electronic Cigarettes

The composition described herein can be present in a cigarette or electronic cigarette. When present in a cigarette, the composition can contact at least one of: (i) the paper, (ii) the tobacco, and (iii) the filter, if present. Such contacting of the composition and cigarette substrate (e.g., paper, tobacco, or filter) will result in the composition being located on the surface of the cigarette substrate, will be at least partially embedded in the cigarette substrate, or a combination thereof.

When present in an electronic cigarette, the composition can be present in the liquid, which is present in the electronic device. The liquid present in the electronic device will typically include nicotine and one or more excipients such as, e.g., propylene glycol, glycerin, and flavoring agent.

Modern commercially manufactured cigarettes are seemingly simple objects consisting mainly of a tobacco blend, paper, glue or adhesive (e.g., PVA) to bond the outer layer of paper together, and often also a filter (e.g., cellulose acetate-based).

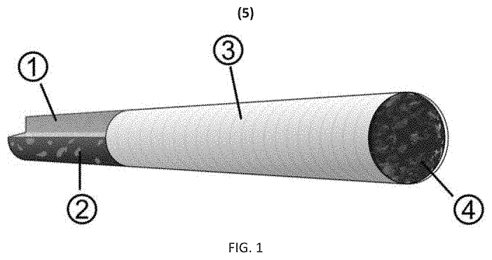

Referring to FIG. 1, a cigarette (5) is provided. The cigarette (5) includes a filter (1), tipping paper (2) that covers the filter (1), tobacco blend (4) and rolling paper (3) that covers the tobacco.

The rolling paper (3) for holding the tobacco blend (4) may vary in porosity to allow ventilation of the burning ember or contain materials that control the burning rate of the cigarette and stability of the produced ash. The tipping paper (2) used in tipping the cigarette (forming the mouthpiece) and surrounding the filter (1) stabilize the mouthpiece from saliva and moderate the burning of the cigarette (5), as well as the delivery of smoke with the presence of one or two rows of small laser-drilled air holes. The composition described herein (not shown) can be present in the cigarette (5), such that the composition contacts any one or more of the filter (1), tipping paper (2), rolling paper (3), and tobacco blend (4).

Modern cigarettes produced after the 1950s, although composed mainly of shredded tobacco leaf, typically use a significant quantity of tobacco processing byproducts in the tobacco blend (4). Each cigarette's tobacco blend (4) is made mainly from the leaves of flue-cured brightleaf, burley tobacco, and oriental tobacco. These leaves are selected, processed, and aged prior to blending and filling. The processing of brightleaf and burley tobaccos for tobacco leaf “strips” produces several byproducts such as leaf stems, tobacco dust, and tobacco leaf pieces (“small laminate”). To improve the economics of producing cigarettes (5), these byproducts are processed separately into forms where they can then be added back into the tobacco blend (4) without an apparent or marked change in the cigarette's (5) quality.

The primary parts that make up an e-cigarette are a mouthpiece, a cartridge (tank), a heating element/atomizer, a microprocessor, a battery, and possibly a LED light on the end. An exception to this is mechanical e-cigarettes (mods) which contain no electronics and the circuit is closed by using a mechanical action switch. An atomizer includes a small heating element, or coil, that vaporizes e-liquid and wicking material that draws liquid onto the coil. When the user pushes a button, or (in some variations) activates a pressure sensor by inhaling, the heating element that atomizes the liquid solution; The e-liquid reaches a temperature of roughly 100-250° C. within a chamber to create an aerosolized vapor. The user inhales the aerosol, commonly called vapor, rather than cigarette smoke. The aerosol provides a flavor and feel similar to tobacco smoking.

There are three main types of e-cigarettes: cigalikes, looking like cigarettes; eGos, bigger than cigalikes with refillable liquid tanks; and mods, assembled from basic parts or by altering existing products. As the e-cigarette industry continues to evolve, new products are quickly developed and brought to market. First generation e-cigarettes tend to look like tobacco cigarettes and so are called “cigalikes”. Most cigalikes look like cigarettes but there is some variation in size. A traditional cigarette is smooth and light while a cigalike is rigid and slightly heavier. Second generation devices are larger overall and look less like tobacco cigarettes. Third generation devices include mechanical mods and variable voltage devices. The fourth generation includes Sub ohm tanks and temperature control devices. The power source is the biggest component of an e-cigarette, which is frequently a rechargeable lithium-ion battery.

In the embodiments below, specific and preferred values and ranges listed for the amount of component of the composition (e.g., vitamin A and/or vitamin E) are provided for illustration and description purposes only; they do not exclude other values or ranges of the component, as defined by the claims. The following values and ranges refer to specific components of the composition.

In specific embodiments, the vitamin A includes at least one of retinol, retinal, retinoic acid, and a provitamin A carotenoid (most notably alpha-carotene, beta-carotene, gamma-carotene, or beta-cryptoxanthin). In specific embodiments, the vitamin A includes retinol. In specific embodiments, the vitamin A is present in up to about 65 wt. % of the composition. In specific embodiments, the vitamin A is present in up to about 70 wt. % of the composition. In specific embodiments, the vitamin A is present in up to about 75 wt. % of the composition. In specific embodiments, the vitamin A is present in up to about 80 wt. % of the composition. In specific embodiments, the vitamin A is present in up to about 85 wt. % of the composition. In specific embodiments, the vitamin A is present in up to about 90 wt. % of the composition. In specific embodiments, the vitamin A is present in up to about 95 wt. % of the composition. In specific embodiments, the vitamin A is present in up to about 98 wt. % of the composition.

In specific embodiments, the vitamin E includes at least one of a tocopherol and a tocotrienol. In specific embodiments, the vitamin E includes a tocopherol. In specific embodiments, the vitamin E includes α-tocopherol. In specific embodiments, the vitamin E includes γ-tocopherol. In specific embodiments, the vitamin E is present in up to about 5 wt. % of the composition. In specific embodiments, the vitamin E is present in up to about 10 wt. % of the composition. In specific embodiments, the vitamin E is present in up to about 15 wt. % of the composition. In specific embodiments, the vitamin E is present in up to about 20 wt. % of the composition. In specific embodiments, the vitamin E is present in up to about 25 wt. % of the composition. In specific embodiments, the vitamin E is present in up to about 30 wt. % of the composition.

In specific embodiments, the vitamin A and vitamin E are present in a weight ratio of at least about 1:1, respectively, of the composition. In specific embodiments, the vitamin A and vitamin E are present in a weight ratio of up to about 50:1, respectively, of the composition. In specific embodiments, the vitamin A and vitamin E are present in a weight ratio of about 1:1 to 50:1, respectively, of the composition. In specific embodiments, the vitamin A and vitamin E are present in a weight ratio of about 1:1 to 20:1, respectively, of the composition. In specific embodiments, the vitamin A and vitamin E are present in a weight ratio of about 1:1 to 10:1, respectively, of the composition. In specific embodiments, the vitamin A and vitamin E are present in a weight ratio of about 10:1 to 50:1, respectively, of the composition. In specific embodiments, the vitamin A and vitamin E are present in a weight ratio of about 10:1 to 40:1, respectively, of the composition. In specific embodiments, the vitamin A and vitamin E are present in a weight ratio of about 10:1 to 30:1, respectively, of the composition. In specific embodiments, the vitamin A and vitamin E are present in a weight ratio of about 5:1 to about 20:1, respectively, of the composition. In specific embodiments, the vitamin A and vitamin E are present in a weight ratio of about 10:1, respectively, of the composition.

In specific embodiments, the composition is administered to the human, such that the vitamin A is administered in an amount of at least about 1 mg/kg of body weight, per day. In specific embodiments, the composition is administered to the human, such that the vitamin A is administered in an amount of up to about 100 mg/kg of body weight, per day. In specific embodiments, the composition is administered to the human, such that the vitamin A is administered up to about 50 mg/kg of body weight, per day. In specific embodiments, the composition is administered to the human, such that the vitamin A is administered up to about 25 mg/kg of body weight, per day. In specific embodiments, the composition is administered to the human, such that the vitamin A is administered up to about 10 mg/kg of body weight, per day. In specific embodiments, the composition is administered to the human, such that the vitamin A is administered in an amount of about 5-20 mg/kg of body weight, per day. In specific embodiments, the composition is administered to the human, such that the vitamin A is administered in an amount of about 10 mg/kg of body weight, per day. In specific embodiments, the composition is administered to the human, such that the vitamin A is administered in an amount of at least about 1 mg/kg of body weight, per day. In specific embodiments, the composition is administered to the human, such that the vitamin A is administered in an amount of at least about 5 mg/kg of body weight, per day. In specific embodiments, the composition is administered to the human, such that the vitamin A is administered in an amount of at least about 10 mg/kg of body weight, per day. In specific embodiments, the composition is administered to the human, such that the vitamin A is administered in an amount of at least about 20 mg/kg of body weight, per day.

In specific embodiments, the composition is administered to the human, such that the vitamin E is administered in an amount of up to about 20 mg/kg of body weight, per day. In specific embodiments, the composition is administered to the human, such that the vitamin E is administered in an amount of up to about 15 mg/kg of body weight, per day. In specific embodiments, the composition is administered to the human, such that the vitamin E is administered in an amount of up to about 10 mg/kg of body weight, per day. In specific embodiments, the composition is administered to the human, such that the vitamin E is administered in an amount of up to about 5 mg/kg of body weight, per day. In specific embodiments, the composition is administered to the human, such that the vitamin E is administered in an amount of up to about 1 mg/kg of body weight, per day. In specific embodiments, the composition is administered to the human, such that the vitamin E is administered in an amount of about 0.5-5.0 mg/kg of body weight, per day. In specific embodiments, the composition is administered to the human, such that the vitamin E is administered in an amount of at least about 0.5 mg/kg of body weight, per day. In specific embodiments, the composition is administered to the human, such that the vitamin E is administered in an amount of at least about 1 mg/kg of body weight, per day. In specific embodiments, the composition is administered to the human, such that the vitamin E is administered in an amount of at least about 2 mg/kg of body weight, per day. In specific embodiments, the composition is administered to the human, such that the vitamin E is administered in an amount of at least about 5 mg/kg of body weight, per day. In specific embodiments, the composition is administered to the human, such that the vitamin E is administered in an amount of at least about 10 mg/kg of body weight, per day.

In specific embodiments, the vitamin A is present in the composition in up to about 500 mg. In specific embodiments, the vitamin A is present in the composition in up to about 250 mg. In specific embodiments, the vitamin A is present in the composition in up to about 100 mg. In specific embodiments, the vitamin A is present in the composition in up to about 75 mg. In specific embodiments, the vitamin A is present in the composition in up to about 50 mg. In specific embodiments, the vitamin A is present in the composition in at least about 1 mg. In specific embodiments, the vitamin A is present in the composition in at least about 5 mg. In specific embodiments, the vitamin A is present in the composition in at least about 10 mg. In specific embodiments, the vitamin A is present in the composition in at least about 25 mg. In specific embodiments, the vitamin A is present in the composition in at least about 50 mg. In specific embodiments, the vitamin A is present in the composition in at least about 75 mg.

In specific embodiments, the vitamin E is present in the composition in up to about 150 mg. In specific embodiments, vitamin E is present in the composition in up to about 75 mg. In specific embodiments, the vitamin E is present in the composition in up to about 50 mg. In specific embodiments, the vitamin E is present in the composition in up to about 25 mg. In specific embodiments, the vitamin E is present in the composition in at least about 1 mg. In specific embodiments, the vitamin E is present in the composition in at least about 5 mg. In specific embodiments, the vitamin E is present in the composition in at least about 10 mg. In specific embodiments, the vitamin E is present in the composition in at least about 15 mg. In specific embodiments, the vitamin E is present in the composition in at least about 25 mg. In specific embodiments, the vitamin E is present in the composition in at least about 50 mg.

In specific embodiments, the vitamin A includes retinol and the vitamin E includes gamma-tocopherol.

In specific embodiments, the composition containing the vitamin A and the vitamin E is present in an inhaler. In specific embodiments, the composition containing the vitamin A and the vitamin E is present in a pressurized metered-dose inhaler (MDI). In specific embodiments, the composition containing the vitamin A and the vitamin E is present in a dry powder inhaler (DPI). In specific embodiments, the composition containing the vitamin A and the vitamin E is present in a nebulizer. In specific embodiments, the composition containing the vitamin A and the vitamin E is present in a nasal inhaler.

### Example

The following example is offered by way of illustration. But the example in no way limits the scope of the embodiments described herein.

In some embodiments, an aerosolizer comprising a composition comprising retinol and vitamin E can deliver a metered dose of retinol of about 300 micrograms upon actuation of the aerosolizer, which is approximately the amount of vitamin A found in about 2 cups of milk. A human subject can take three doses of the compositions of the various embodiments described herein (e.g., a dose in the morning noon, and in the evening). This would provide a total of about 900 micrograms of retinola per day. Even if the human subject were to administer two metered doses three times a day, that would yield 1800 micrograms of retinol per day, which is well under the tolerable upper limit of 3000 micrograms. A daily amount of retinol of about 900 micrograms to about 1800 micrograms should be an effective dose to protect the lung, among other organs, against damage due to retinol deficiency. Such daily amounts of retinol should be sufficient to maintain adequate levels of vitamin A in the lung, among other organs.

With regard to vitamin E, the amount of vitamin E delivered per metered dose is about 10 mg alpha-tocopherol per aerosolizer actuation and about 10 mg gamma-tocopherol per actuation. Three metered doses per day will yield about 30 mg of each form of vitamin E and 60 mg total of vitamin E. It is believed that since the vitamin E, as well as the retinol, is being delivered into the lung, the dose does not have to be in a pharmacological range. This still would fall within a physiological amount. It is four times the daily recommended amount for adults (RDA for adults is 15 mg/day). Vitamin E is fairly nontoxic, however, over 1000 mg could interfere with vitamin K and prevent clotting and increase risk for stroke. We are well below this toxic amount.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those of ordinary skill in the art, and that such modifications and variations are considered to be within the scope of this invention as defined by the appended claims.

### Enumerated Embodiments

Enumerated embodiments [1] to [39] provided below are for illustration purposes only, and do not otherwise limit the scope of the embodiments described herein, as encompassed by the claims. These enumerated embodiments encompass all combinations, sub-combinations, and multiply referenced (e.g., multiply dependent) combinations described therein.

[1] An embodiment relates to a composition that includes: (i) vitamin A (e.g., retinol), and (ii) vitamin E (e.g., gamma-tocopherol), that is suitable for at least one of pulmonary administration, aerosolization, and intranasal administration.
[2] Another embodiment relates to the composition of embodiment [1], which is in a solid, powder form, suitable for pulmonary administration.
[3] Another embodiment relates to the composition of embodiment [1], which is in a liquid form, suitable for aerosolization.
[4] Another embodiment relates to the composition of embodiment [1], which is in a liquid form, suitable for intranasal administration.
[5] Another embodiment relates to the composition of embodiment [1], which is in a liquid form, suitable for pulmonary administration.
[6] Another embodiment relates to the composition of any one of embodiments [1]-[5], further including a pharmaceutically acceptable carrier.
[7] Another embodiment relates to the composition of any one of embodiments [1]-[6], further including one or more pharmaceutically acceptable excipients.
[8] Another embodiment relates to the composition of any one of embodiments [1]-[7], wherein the vitamin A (e.g., retinol) is present in up to about 95 wt. % of the composition.
[9] Another embodiment relates to the composition of any one of embodiments [1]-[8], wherein the vitamin E (e.g., gamma-tocopherol) is present in up to about 15 wt. % of the composition.
[10] Another embodiment relates to the composition of any one of embodiments [1]-[9], wherein the vitamin A (e.g., retinol) and vitamin E (e.g., gamma-tocopherol) are present in a weight ratio of about 10:1, respectively.
[11] Another embodiment relates to the composition of any one of embodiments [1]-[10], wherein the vitamin A (e.g., retinol) and vitamin E (e.g., gamma-tocopherol) are present in a weight ratio of about 5:1 to about 20:1, respectively.
[12] Another embodiment relates to a method that includes administering to a human an effective amount of the composition of any one of embodiments [1]-[11].
[13] Another embodiment relates to the method of embodiment [12], wherein the human is at least 18 years old.
[14] Another embodiment relates to the method of any one of embodiments [12]-[13], wherein the human currently smokes tobacco, such that the human currently engages in the practice of taking tobacco smoke into the mouth, and then releasing it.
[15] Another embodiment relates to the method of any one of embodiments [12]-[14], wherein the human has previously smoked tobacco, such that the human has previously engaged in the practice of taking tobacco smoke into the mouth, and then releasing it.
[16] Another embodiment relates to the method of any one of embodiments [12]-[15], which is a method of protecting the human against one or more tobacco smoke pathologies.
[17] Another embodiment relates to the method of any one of embodiments [12]-[16], which is a method of decreasing in the human, the occurrence of one or more diseases caused by tobacco smoking.
[18] Another embodiment relates to the method of any one of embodiments [12]-[17], which is a method of decreasing in the human, the progression of one or more diseases caused by tobacco smoking.
[19] Another embodiment relates to the method of any one of embodiments [12]-[18], wherein the human is afflicted with, or is at risk of developing, one or more of: emphysema, COPD, bronchitis, pneumonitis or pulmonitis, lung cancer, oral cancer, tongue cancer, and heart disease, at least partially caused by tobacco smoking.
[20] Another embodiment relates to the method of any one of embodiments [12]-[19], wherein the vitamin A (e.g., retinol) is administered to the human in an amount of about 10 mg/kg of body weight, per day.
[21] Another embodiment relates to the method of any one of embodiments [12]-[20], wherein the vitamin E (e.g., gamma-tocopherol) is administered to the human in an amount of about 1 mg/kg of body weight, per day.
[22] Another embodiment relates to the method of any one of embodiments [12]-[21], wherein the human has been diagnosed with one or more of emphysema, COPD, bronchitis, lung cancer, and heart disease.
[23] Another embodiment relates to the method of any one of embodiments [12]-[22], wherein the human is at risk of one or more of emphysema, COPD, bronchitis, lung cancer, and heart disease.
[24] Another embodiment relates to the method of any one of embodiments [12]-[23], wherein the administration is within the oral cavity, the respiratory system, or a combination thereof.
[25] Another embodiment relates to a nutritional supplement that includes the composition of any one of embodiments [1]-[11], in a unit dosage form or a metered dosage form.
[26] Another embodiment relates to an intranasal spray that includes the composition of any one of embodiments [1]-[11] and a pharmaceutically acceptable liquid carrier, which is suitable for intranasal administration.
[27] Another embodiment relates to a pulmonary spray that includes the composition of any one of embodiments [1]-[11] and a pharmaceutically acceptable liquid carrier, which is suitable for pulmonary administration.
[28] Another embodiment relates to a pulmonary powder that includes the composition of any one of embodiments [1]-[11] and a pharmaceutically acceptable solid carrier, which is suitable for pulmonary administration.
[29] Another embodiment relates to a cigarette that includes the composition of any one of embodiments [1]-[11], suitable for pulmonary administration.
[30] Another embodiment relates to an electronic cigarette that includes: a handheld electronic device configured to effectively vaporize a flavored liquid suitable for pulmonary administration, wherein the flavored liquid includes the composition of any one of embodiments [1]-[11], and one or more of nicotine, propylene glycol, glycerin, cannabinoids, and a flavoring agent.
[31] Another embodiment relates to the electronic cigarette of embodiment [30], including a mouthpiece, a cartridge (tank), a heating element/atomizer, a microprocessor, a battery, and optionally a LED light.
[32] Another embodiment relates to a cigarette that includes: (i) tobacco leaves dried to a water content of less than about 5 wt. %, (ii) rolling paper, (iii) optionally a cigarette filter, and (iv) the composition of any one of embodiments [1]-[11], which is in contact with at least one of the dried tobacco leaves, the rolling paper, and the cigarette filter, wherein the rolling paper substantially forms a cylinder with the dried tobacco leaves located inside the cylinder and wherein the cigarette filter, when present, is located at one terminus of the cylinder.
[33] Another embodiment relates to the cigarette of embodiment [32], wherein the vitamin A (e.g., retinol) is present in up to about 250 mg.
[34] Another embodiment relates to the cigarette of any one of embodiments [32]-[33], wherein the vitamin E (e.g., gamma-tocopherol) is present in up to about 75 mg.
[35] Another embodiment relates to the cigarette of any one of embodiments [32]-[34], having a length of about 100 mm, a diameter of about 8 mm, and a length of filter of about 20 mm.
[36] Another embodiment relates to a cigarette that includes: (i) marijuana leaves dried to a water content of less than about 5 wt. %, (ii) rolling paper, (iii) optionally a cigarette filter, and (iv) the composition of any one of embodiments [1]-[11], which is in contact with at least one of the dried marijuana leaves, the rolling paper, and the cigarette filter, wherein the rolling paper substantially forms a cylinder with the dried marijuana leaves located inside the cylinder and wherein the cigarette filter, when present, is located at one terminus of the cylinder.
[37] Another embodiment relates to a method that includes: (i) igniting a terminus of the cigarette of any one of embodiments [32]-[36], the terminus including at least one of the rolling paper and the dried tobacco, the terminus not including the cigarette filter, (ii) directing tobacco smoke from the ignited cigarette into the mouth, and (iii) releasing the tobacco smoke, wherein the presence of the composition of any one of embodiments [1]-[11] decreases the occurrence of one or more diseases caused by tobacco smoking.
[38] Another embodiment relates to the method of embodiment [37], wherein the directing of the tobacco smoke from the ignited cigarette into the mouth is followed by inhaling the tobacco smoke.
[39] Another embodiment relates to the method of any one of embodiments [37]-[38], wherein the releasing of the tobacco smoke is associated with exhaling the tobacco smoke.

### REFERENCES

1. U.S. Department of Health and Human Services. The Health Consequences of Smoking-50 Years of Progress: A Report of the Surgeon General. Atlanta: U.S. Department of Health and Human Services, Centers for Disease Control and Prevention, National Center for Chronic Disease Prevention and Health Promotion, Office on Smoking and Health, 2014.
2. U.S. Department of Health and Human Services. How Tobacco Smoke Causes Disease: What It Means to You. Atlanta: U.S. Department of Health and Human Services, Centers for Disease Control and Prevention, National Center for Chronic Disease Prevention and Health Promotion, Office on Smoking and Health, 2010.
3. Centers for Disease Control and Prevention. QuickStats: Number of Deaths from 10 Leading Causes-National Vital Statistics System, United States, 2010. Morbidity and Mortality Weekly Report 2013:62(08); 155.
4. U.S. Department of Health and Human Services. Reducing the Health Consequences of Smoking: 25 Years of Progress. A Report of the Surgeon General. Rockville (Md.): U.S. Department of Health and Human Services, Public Health Service, Centers for Disease Control, National Center for Chronic Disease Prevention and Health Promotion, Office on Smoking and Health, 1989 [accessed 2015 Oct. 5].
5. Benowitz N L; Benowitz, Neal L. (2010). “Nicotine addiction”. N Engl J Med 362 (24): 2295-303. doi:10.1056/NEJMra0809890. PMC 2928221. PMID 20554984.

What is claimed is:

1. A method for treating a human subject afflicted with, or is at risk of developing, one or more of:
lung cancer,
oral cancer, and
tongue cancer
the method comprising administering to the human a composition:
(i) at least one of retinol and retinoic acid;
(ii) vitamin E; and
(iii) a pharmaceutically acceptable carrier suitable for pulmonary administration.

2. The method of claim 1, wherein the retinol or retinoic acid is administered to the human in an amount of about 10 mg/kg of body weight, per day.

3. The method of claim 1, wherein the vitamin E is administered to the human in an amount of about 1 mg/kg of body weight, per day.

4. The method of claim 1, wherein the administration is within the oral cavity, the respiratory system, or a combination thereof.

5. The method of claim 1, wherein the retinol or retinoic acid is present in up to about 95 wt. % of the composition.

6. The method of claim 1, wherein the vitamin E is present in up to about 15 wt. % of the composition.

7. The method of claim 1, wherein the retinol or retinoic acid and vitamin E are present in a weight ratio of about 10:1, respectively.

8. The method of claim 1, wherein the retinol or retinoic acid and vitamin E are present in a weight ratio of about 5:1 to about 20:1, respectively.

9. The method of claim 1, wherein the composition is in a liquid form and suitable for aerosolized administration to the lung.

10. A method for treating a human subject afflicted with, or is at risk of developing, one or more of:
emphysema,
COPD,
pneumonitis or pulmonitis, and
asthma,
the method comprising administering to the human a composition:
(i) at least one of retinol and retinoic acid;
(ii) vitamin E; and
(iii) a pharmaceutically acceptable carrier suitable for pulmonary administration.

11. The method of claim 10, wherein the retinol or retinoic acid is administered to the human in an amount of about 10 mg/kg of body weight, per day.

12. The method of claim 10, wherein the vitamin E is administered to the human in an amount of about 1 mg/kg of body weight, per day.

13. The method of claim 10, wherein the administration is within the oral cavity, the respiratory system, or a combination thereof.

14. The method of claim 10, wherein the retinol or retinoic acid is present in up to about 95 wt. % of the composition.

15. The method of claim 10, wherein the vitamin E is present in up to about 15 wt. % of the composition.

16. The method of claim 10, wherein the retinol or retinoic acid and vitamin E are present in a weight ratio of about 10:1, respectively.

17. The method of claim 10, wherein the retinol or retinoic acid and vitamin E are present in a weight ratio of about 5:1 to about 20:1, respectively.

18. The method of claim 10, wherein the composition is in a liquid form and suitable for aerosolized administration to the lung.
